(19) United States
(12) Patent Application Publication
(10) Pub. No.: US 2011/0035010 A1
(43) Pub. Date: Feb. 10, 2011

(54) TOROID-SHAPED SPINAL DISC

(75) Inventor: Scott Harrington, Westwood, NJ (US)

Correspondence Address: HARNESS, DICKEY & PIERCE, P.L.C., P.O. BOX 828, BLOOMFIELD HILLS, MI, 48303, US

(73) Assignee: EBI, LLC, Parsippany, NJ (US)

(21) Appl. No.: 12/840,623

(22) Filed: Jul. 21, 2010

Related U.S. Application Data
(63) Continuation-in-part of application No. 12/839,491, filed on Jul. 20, 2010.
(60) Provisional application No. 61/232,208, filed on Aug. 7, 2009.

(51) Int. Cl. A61F 2/44

(52) U.S. Cl. 623/17.16

(57) ABSTRACT

An intervertebral implant for insertion between adjacent vertebral bodies is provided. The intervertebral implant can include a first component including a first articulating surface, which can be generally convex with a first radius of curvature. The first articulating surface can also define a stop element. The intervertebral implant can comprise a second component defining an aperture and including a second articulating surface. The second articulating surface can be generally concave and can have a second radius of curvature. The second articulating surface can be articulable with the first articulating surface for retaining motion between the first and second vertebra. A portion of the first articulating surface including the stop element can extend into the aperture and contacts a sidewall of the aperture to limit the range of articulation of the first component and the second component.

### CROSS-REFERENCE TO RELATED APPLICATIONS

This application claims the benefit of U.S. Provisional Application No. 61/232,208, filed on Aug. 7, 2009. The entire disclosure of the above application is incorporated herein by reference. In addition, this application is a continuation-in-part of U.S. patent application Ser. No. 12/839,491 filed on Jul. 20, 2010. The entire disclosure of this application is also incorporated herein by reference.

### INTRODUCTION

The spinal column provides the main support for the body and is made of thirty-three individual bones called vertebrae. There are twenty-four moveable vertebrae in the spine, while the remaining vertebrae are fused. Each individual vertebra can include a posterior vertebral arch for protecting the spinal cord, posterior processes extending from the vertebral arch, and an anterior, drum-shaped vertebral body having superior and inferior endplates. The vertebral body can transmits loads to adjacent bodies via an anterior intervertebral disc and two posterior facets.

The moveable vertebrae are stacked in series and are separated and cushioned by the anterior intervertebral discs. Each intervertebral disc is composed of an outer fibrous ring (i.e., annulus) operating as a pseudo pressure vessel for retaining an incompressible fluid (i.e., nucleus pulposus). The nucleus pulposus is a gel-like substance housed centrally within the annulus and sandwiched between the endplates of the adjacent vertebral bodies. In a healthy disc, the nucleus pulposus acts as a hard sphere seated within the nuclear recess (i.e., fossa) of the vertebral endplates. This sphere operates as the fulcrum (i.e., nuclear fulcrum) for mobility in the spine. Stability is achieved by balancing loads in the annulus and the facet joints.

Degenerative disc disease (DDD) affects the physiology of the disc and may be caused by aging, trauma, or various other factors. DDD results in a reduction in disc height, which in turn, alters the loading pattern in the facets. This altered loading pattern may cause symptomatic degeneration of the facet joints, which may reduce stability and compress the nerves branching out of the spinal column.

Examples of surgical treatments for DDD include spinal fusion and total disc arthroplasty. Total disc arthroplasty may be used to preserve anatomical motion between adjacent vertebral bodies, may reduce stress sustained by adjacent spinal levels, and may slow down disc degeneration.

### SUMMARY

The present teachings provide a toroid-shaped spinal disc and more particularly, a toroid-shaped spinal disc having superior and inferior components mutually articulating to replicate natural spine movement.

According to one aspect, an intervertebral implant for insertion between adjacent vertebral bodies is provided. The intervertebral implant can comprise a first component including a first articulating surface, which can be generally convex with a first radius of curvature. The first articulating surface can also define a stop element. The intervertebral implant can comprise a second component defining an aperture and including a second articulating surface. The second articulating surface can be generally concave and can have a second radius of curvature. The second articulating surface can be articulable with the first articulating surface for retaining motion between the first and second vertebra. A portion of the first articulating surface including the stop element can extend into the aperture and contacts a sidewall of the aperture to limit the range of articulation of the first component and the second component.

According to a further aspect, an intervertebral implant is provided. The intervertebral implant can include a first component having a first articulating surface, which can be generally convex. The intervertebral implant can include a second component defining an aperture, which can have a second articulating surface and a first bone engaging portion for engaging a first vertebra. The second articulating surface can be generally concave and articulable with the first articulating surface for retaining motion between the first vertebra and a second vertebra. The intervertebral implant can also include a third component coupled to the first component opposite the first articulating surface. The third component can define a second bone engaging portion for engaging the second vertebra. The second articulating surface can have a larger radius of curvature than the first articulating surface such that a portion of the first articulating surface extends into the aperture of the second component.

Also provided is an intervertebral implant, which can comprise a first component having a first articulating surface. The first articulating surface can be generally convex. The intervertebral implant can also comprise a second component in the shape of a toroid that can define an aperture. The second component can have a second articulating surface, which can be generally concave and articulable with the first articulating surface for retaining motion between a first vertebra and a second vertebra. The intervertebral implant can also include a third component coupled to the first component opposite the first articulating surface. The third component can define a first bone engaging portion for engaging a first vertebra. The intervertebral implant can include a fourth component coupled to the second component opposite the second articulating surface so as to be disposed about a periphery of the aperture. The fourth component can define a second bone engaging portion. The second articulating surface can have a larger radius of curvature than the first articulating surface such that a portion of the first articulating surface extends into the aperture of the second component.

Further areas of applicability will become apparent from the description provided herein. It should be understood that the description and specific examples are intended for purposes of illustration only and are not intended to limit the scope of the present teachings.

### DRAWINGS

The present invention will become more fully understood from the detailed description and the accompanying drawings, wherein:

FIG. 1 is a schematic sagittal view of an intervertebral implant according to the present teachings, the intervertebral implant shown implanted in a spine.

FIG. 2 is a perspective view of the intervertebral implant according to the present teachings.

FIG. 2A is a perspective view of the intervertebral implant of FIG. 2.

FIG. 3 is an end view of the intervertebral implant of FIG. 2.

FIG. 4 is a perspective view of the intervertebral implant of FIG. 2.

FIG. 5 is a sectional view taken along the line 5-5 of FIG. 4.

FIG. 6 is a sectional view taken along the line 6-6 of FIG. 4.

FIG. 6A is a cross-sectional view of an alternative intervertebral implant according to the present teachings.

FIG. 7 is a partial sectional view illustrating another intervertebral implant in accordance with the present teachings.

FIG. 8 is another partial sectional view of the intervertebral implant of FIG. 7.

FIG. 9 is a schematic illustration of one of various intervertebral implants according to the present teachings.

FIG. 10 is an exploded side view of the intervertebral implant of FIG. 9.

FIG. 11 is an exploded perspective view of the intervertebral implant of FIG. 9.

FIG. 12 is a sectional view taken along the line 12-12 of FIG. 9.

FIG. 13 is a perspective view of one of various intervertebral implants according to the present teachings.

FIG. 14 is an exploded view of the intervertebral implant of FIG. 13.

FIG. 14A is a perspective view of an inferior component associated with the intervertebral implant of FIG. 13.

FIG. 15 is a cross-sectional view of the intervertebral implant of FIG. 13, taken along line 15-15 of FIG. 13.

FIG. 16 is a perspective view of one of various intervertebral implants according to the present teachings.

FIG. 17 is an exploded view of the intervertebral implant of FIG. 16.

FIG. 18 is a cross-sectional view of the intervertebral implant of FIG. 16, taken along line 18-18 of FIG. 16.

### DESCRIPTION OF VARIOUS ASPECTS

The following description is merely exemplary in nature and is not intended to limit the present teachings, application, or uses. It should be understood that throughout the drawings, corresponding reference numerals indicate like or corresponding parts and features. Although the following description is related generally to a method and apparatus for use in an anatomy to repair damaged tissue, such as in the case of degenerative disc disease (DDD), it will be understood that the method and apparatus as described and claimed herein, can be used in any appropriate surgical procedure, such as in a spinal fixation or fusion procedure. Therefore, it will be understood that the following discussions are not intended to limit the scope of the present teachings and claims herein.

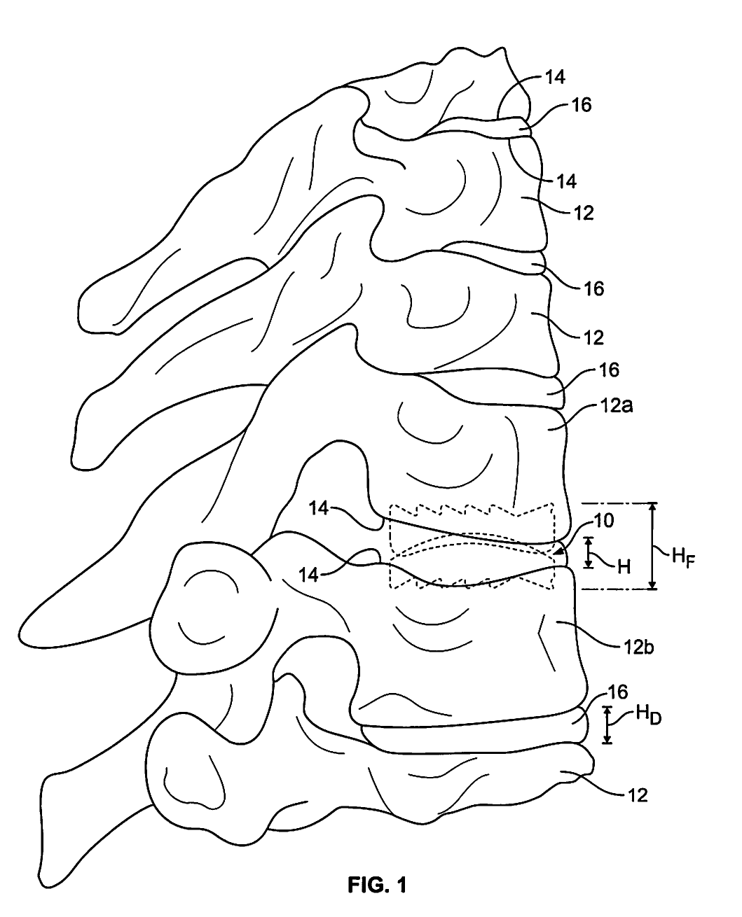

Referring to the environmental view of FIG. 1, an exemplary intervertebral implant 10 according to the present teachings is illustrated as positioned or implanted between two adjacent vertebral bodies 12 of a spine. Generally, the intervertebral implant 10 can be positioned between endplates 14 of the vertebral bodies 12 to replace a degenerative disc. In certain applications, the intervertebral implant 10 can be positioned between adjacent vertebral bodies 12 in a cervical region of the spine, however, the intervertebral implant 10 can be used in other anatomical locations, such as the lumbar or thoracic spine. Although a single intervertebral implant 10 is illustrated and described herein as being positioned between a single pair of adjacent vertebral bodies 12, it should be understood that any number of intervertebral implants 10 could be positioned between any suitable pair of vertebral bodies 12. As will be discussed herein, the intervertebral implant 10 can be shaped such that the intervertebral implant 10 provides mutually articulating motion at a reduced implant height, which can allow for more natural motion of a spine of a patient.

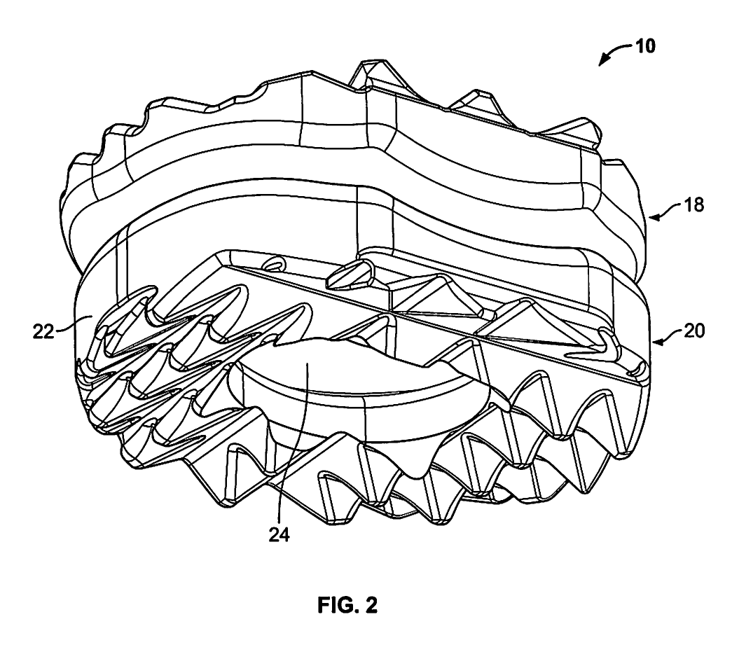

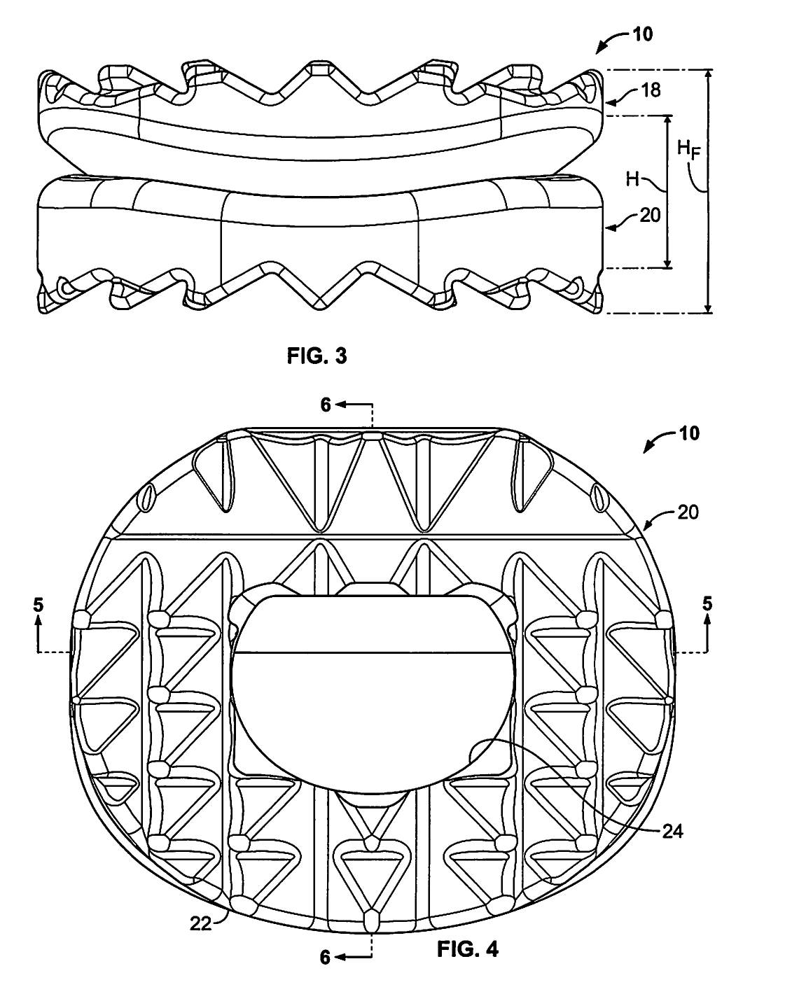

With additional reference to FIGS. 2-6, the intervertebral implant 10 can include multiple components configured for mutual articulation to enable anatomical motion between two adjacent vertebral bodies 12. As illustrated in this example, the intervertebral implant 10 can include a first or inferior component 18 and a second or superior component 20. As will become more apparent below, the inferior component 18 and the superior component 20 can be positioned between adjacent vertebral bodies 12, and can be sized to re-establish a disc height H_{D} associated with a healthy disc 16 to its original dimension. Thus, improved motion and increased stability of the spine may be established.

With continued reference to FIGS. 2-6, at least one of the first and second components 18, 20 can have a generally toroidal shape. As used herein, the phrase “generally toroidal shape” and “generally toroid” shall mean a shape having a main body 22 defining a substantially closed perimeter and an opening or aperture 24. The aperture 24 can be a generally central opening, insofar as it is surrounded by the main body 22. As will be discussed in greater detail herein, in the example of FIGS. 2-6, the superior component 20 can have the generally toroidal shape. In other applications, however, the inferior component 18 can additionally or alternatively have the generally toroidal shape.

The inferior component 18 can comprise an integral component, which can be composed of a suitable biocompatible material, such as a biocompatible metal or polymer. For example, the inferior component 18 can be composed of titanium, cobalt chromium, stainless steel, pyrolytic carbon, etc. If desired, the inferior component 18 can be coated with a suitable biocompatible coating, such as an antibiotic, bone growth material, etc. It should be noted that although the inferior component 18 is described and illustrated herein as comprising a single integral component, the inferior component 18 could comprise multiple components coupled together, if desired. For example, the inferior component 18 could comprise a biocompatible polymer, such as polyethylene, coupled to a biocompatible metal, such as titanium, through a suitable technique. The inferior component 18 can include a first articulating surface 26 and a first bone engagement surface 28. Generally, the first articulating surface 26 can be positioned opposite the first bone engagement surface 28. As will be discussed in greater detail herein, the first articulating surface 26 can cooperate with the superior component 20 to enable relative motion between the inferior component 18 and the superior component 20.

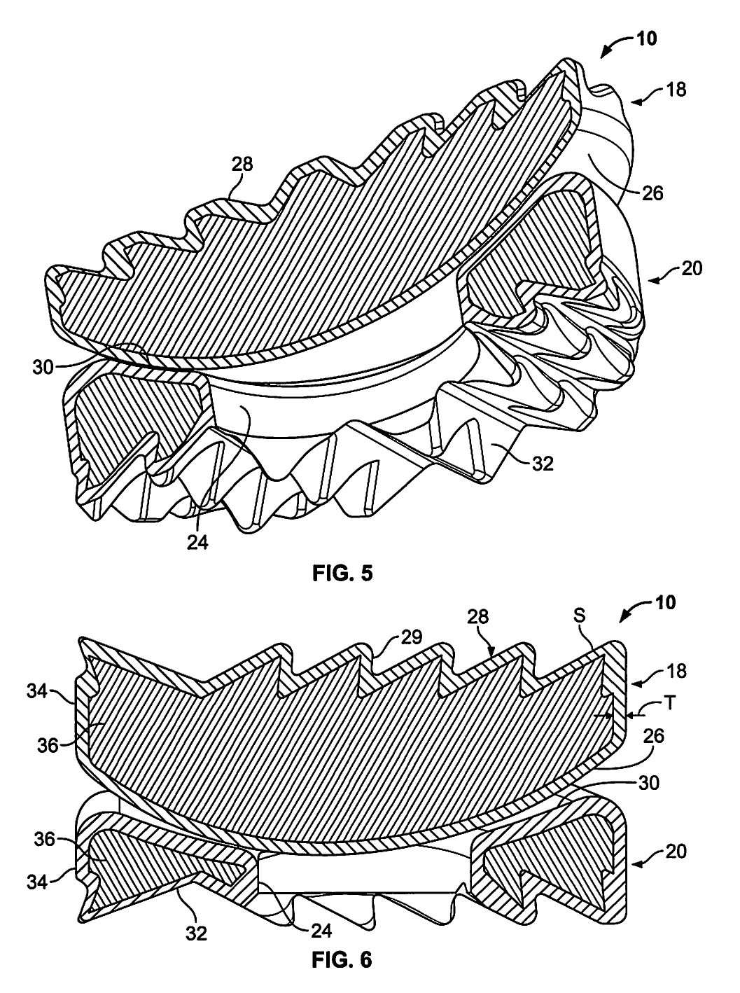

In one example, the first articulating surface 26 can be convex, concave or combinations thereof. In the example of FIGS. 2-6, the first articulating surface 26 can be generally convex. In this regard, as best shown in FIG. 5, the first articulating surface 26 can be substantially hemispherical, and can include a first radius of curvature. It should be noted, however, that the first articulating surface 26 can have any shape that enables motion between the inferior component 18 and the superior component 20. For example, the first articulating surface 26 can include distinct radii of curvature that may or may not be concentric.

With reference to FIG. 1, the first bone engagement surface 28 can engage a first vertebra or vertebral body 12a. The first bone engagement surface 28 may be configured in any manner well known in the art to resist expulsion of the intervertebral implant 10 from between the adjacent vertebral bodies 12, and to enable the inferior component 18 to self-center or self-align relative to the vertebral body 12a. In one example, with reference to FIGS. 2-6, the first bone engagement surface 28 can include aggressive multi-angled and self-centering teeth 29 for fixation. The particular structure of the first bone engagement surface 28 will be understood to be beyond the scope of the present teachings.

Briefly, however, with reference to FIG. 2A, the teeth 29 of the first bone engagement surface 28 can each include an elongate angled surface T1, which can terminate at a distal point. The distal point can bite into or alter the surface of the vertebral body 12a to couple or fix the inferior component 18 to the vertebral body 12a (FIG. 1). In one example, as shown in FIG. 2A, the various elongate angled surfaces T1 of the teeth 29 can be arranged so as to enable the inferior component 18 to self-center under loads from the adjacent vertebral bodies 12. In this example, the elongate angled surfaces T1 of a first sub-plurality 29a of the teeth 29 can extend in a first direction D1, a second sub-plurality 29b of the teeth 29 can extend in a second direction D2, and a third sub-plurality 29c of the teeth 29 can extend in a third direction D3. Each of the first direction D1, second direction D2 and third direction D3 can be substantially different and can each be directed away from an end 18a of the inferior component 18. The substantially distinct directions of the sub-pluralities 29a, 29b, 29c of the teeth 29 can enable the inferior component 18 to self-center or self-align with the vertebral body 12a. In addition, the inferior component 18 can include a fourth sub-plurality 29d of teeth 29, which can prevent the expulsion of the inferior component 18.

With reference to FIGS. 2-6, the superior component 20 can comprise an integral component, which can be composed of a suitable biocompatible material, such as a biocompatible metal or polymer. For example, the superior component 20 can be composed of titanium, cobalt chromium, stainless steel, pyrolytic carbon, etc. If desired, the superior component 20 can be coated with a suitable biocompatible coating, such as an antibiotic, bone growth material, etc. The superior component 20 can be composed of the same material as the inferior component 18, or can be composed of a different material than the inferior component 18, depending upon desired strength properties, wear properties, etc. It should be noted that although the superior component 20 is described and illustrated herein as comprising a single integral component, the superior component 20 could comprise multiple components coupled together, if desired. For example, the superior component 20 could comprise a biocompatible polymer, such as polyethylene, coupled to a biocompatible metal, such as titanium, through a suitable technique. The superior component 20 can be generally toroidal in shape, and can include a second articulating surface 30, a second bone engagement surface 32 and the aperture 24.

The second articulating surface 30 may be of any shape to cooperate with the first articulating surface 26 to enable relative motion between the inferior component 18 and the superior component 20. Thus, the second articulating surface 30 can comprise any surface that can cooperate with the first articulating surface 26 to enable relative motion between the vertebral bodies 12. In this example, as the first articulating surface 26 can have a generally convex shape, the second articulating surface 30 can have a generally concave shape. It should be noted, however, that the first articulating surface 26 could comprise a generally concave shape, and the second articulating surface 30 could comprise a generally convex shape, if desired. In this example, the second articulating surface 30 can have a generally hemispherical surface, which can define a second radius of curvature. It should be noted, however, that the second articulating surface 30 can have any shape that enables motion between the inferior component 18 and the superior component 20. For example, the second articulating surface 30 could comprise distinct radii of curvature that may or may not be concentric.

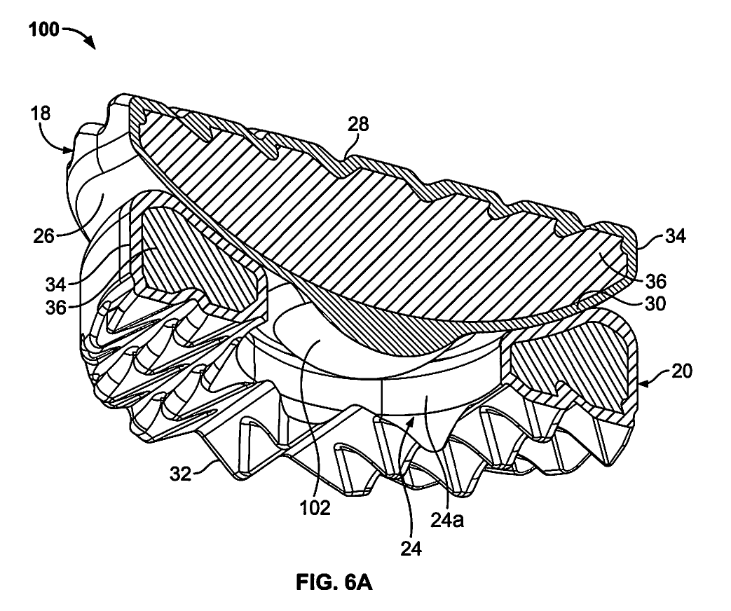

In addition, as best shown in FIGS. 5 and 6, the second radius of curvature can be greater than the first radius of curvature, which can establish line contact between the first articulating surface 26 and the second articulating surface 30 of the inferior component 18 and the superior component 20. The line contact may be generally arcuate due to the generally hemispherical surfaces of each of the inferior component 18 and the superior component 20. The line contact between the inferior component 18 and the superior component 20 can maintain stable articulation between the inferior component 18 and the superior component 20.

Further, if the second radius of curvature associated with the superior component 20 is greater than the first radius of curvature associated with the inferior component 18, a portion of the first articulating surface 26 can extend into the aperture 24 of the superior component 20. In this manner, the profile of the intervertebral implant 10 may be reduced without compromising the performance of the intervertebral implant 10.

With reference back to FIGS. 1-6, the second bone engagement surface 32 can engage a second vertebra or vertebral body 12b. The second bone engagement surface 32 may be configured in any manner well known in the art to resist expulsion of the intervertebral implant 10 from between the adjacent vertebral bodies 12, and to enable the superior component 20 to self-center or self-align relative to the vertebral body 12b. As the second bone engagement surface 32 can be similar to the first bone engagement surface 28 described with regard to the inferior component 18, the second bone engagement surface 32 will not be discussed in great detail herein, and the same reference numerals will be used to denote the same or similar components. Briefly, however, in this example, the second bone engagement surface 32 can include the teeth 29, which can self-center or self-align the superior component 20 relative to the vertebral body 12b, while resisting the explusion of the superior component 20. It should be noted that the second bone engagement surface 32 can include any suitable bone engagement surface known in the art, such as spikes, barbs, etc.

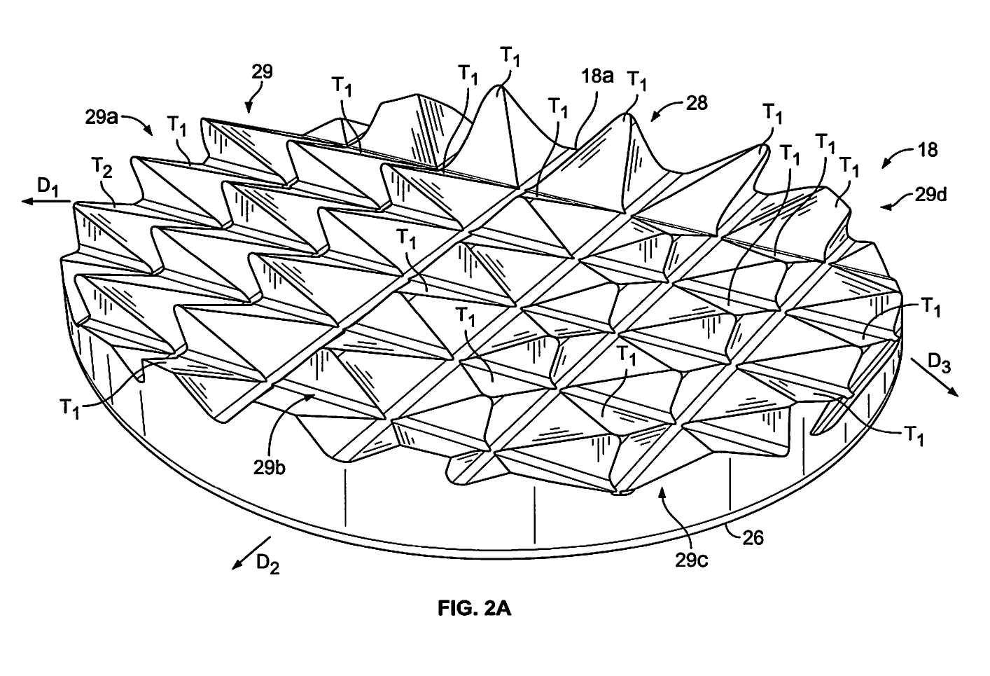

With reference to FIGS. 3-6, the aperture 24 can be formed through the superior component 20 so as to extend from the second articulating surface 30 to the second bone engagement surface 32. The aperture 24 can generally receive a portion of the first articulating surface 26 of the inferior component 18, which can reduce an implant height H of the intervertebral implant 10, as will be discussed in greater detail herein (FIG. 3). It will be understood, however, that the aperture 24 need not extend through the superior component 20. In this regard, the aperture 24 can extend through only a portion of the superior component 20. In the case of the aperture 24 extending only partially through the superior component 20, the aperture 24 may intersect the second articulating surface 30, but may extend only substantially through the superior component 20. In other words, the aperture 24 can be formed as a depression within the second articulating surface 30 so that the aperture 24 can receive the first articulating surface 26 of the inferior component 18 to reduce the height H of the intervertebral implant 10, but the aperture 24 need not extend all the way through the superior component 20.

In one of various examples, with particular reference to FIGS. 5 and 6, at least one of the inferior component 18 and the superior component 20 can comprise a shell 34 and an inner core 36. By constructing at least one of the inferior component 18 and the superior component 20 to have a shell 34 and an inner core 36, the inferior component 18 and/or superior component 20 can have increased wear properties while providing a degree of compressability. In other words, the use of a shell 34 and an inner core 36 can provide the benefits of two materials through a single integral component.

In this regard, the shell 34 can be constructed of a first material having a first hardness. As the shell 34 can form an outer surface S of the inferior component 18 and/or the superior component 20, the shell 34 can have a suitable hardness that enables the inferior component 18 and/or the superior component 20 to engage the vertebral bodies 12 and articulate relative to each other. In addition, the shell 34 can have a thickness T. The thickness T of the shell 34 can coordinate with the hardness of the shell 34 to facilitate the desired wear characteristics and to enable a degree of compressability for the inferior component 18 and/or the superior component 20. It can be desirable to have a degree of compressibility for the inferior component 18 and/or the superior component 20 as it enables the patient to undergo some flexion of the spine, thereby providing the patient with more natural motion.

With continued regard to FIGS. 5 and 6, the inner core 36 can be constructed of a second material having a second hardness. As the inner core 36 can be wholly retained within the shell 34, the second hardness of the inner core 36 can be distinct from the first hardness of the shell 34. In this example, the first hardness of the shell 34 can be greater than the second hardness of the inner core 36. For example, the shell 34 can be constructed of pyrolytic carbon and the inner core 36 can be constructed of graphite. The use of the pyrolytic carbon for the shell 34 can protect the inferior component 18 and/or the superior component 20 from wear, while the use of graphite for the inner core 36 can provide a degree of compressibility for the inferior component 18 and/or the superior component 20.

In the example of FIGS. 2-6, each of the inferior component 18 and the superior component 20 can include the shell 34 and the inner core 36, however, it will be understood that only one of the inferior component 18 or the superior component 20 or none of the inferior component 18 and the superior component 20 could include the shell 34 and inner core 36.

With reference to FIGS. 5 and 6, in order to assemble the intervertebral implant 10, the inferior component 18 can be aligned with the superior component 20 such that the first articulating surface 26 is at least partially received within the aperture 24, and the first articulating surface 26 is in contact with the second articulating surface 30. Then, with the intervertebral implant 10 assembled, the intervertebral implant 10 can be inserted into the anatomy. As the insertion of the intervertebral implant 10 is generally well known in the art, the insertion of the intervertebral implant 10 will not be discussed in great detail herein. Briefly, however, in order to insert the intervertebral implant 10 into the anatomy, such as between adjacent vertebral bodies 12 (FIG. 1), the anatomy can be prepared to receive in the intervertebral implant 10. In this regard, surgical access can be made to an area adjacent to the vertebral bodies 12. For example, surgical access can be obtained via a minimally invasive surgical procedure or a posterior unilateral open procedure.

With access gained to the surgical site, the surgical site can be prepared to receive the intervertebral implant 10. Then, the intervertebral implant 10 can be coupled to a suitable instrument, which can guide the intervertebral implant 10 into the space defined between the adjacent vertebral bodies 12. With the intervertebral implant 10 properly positioned between the vertebral bodies 12, the intervertebral implant 10 can restore the space between the adjacent vertebral bodies 12 to a height substantially similar to the height H_{D} of a healthy disc 16.

In this regard, with reference to FIG. 1, when the intervertebral implant 10 is positioned between adjacent vertebral bodies 12, the implant height H of the intervertebral implant 10 can be substantially similar to the height H_{D} of a healthy disc 16 so as to restore substantially normal function to the spine of the patient. In one example, the implant height H_{D} of the intervertebral implant 10 can range from about 4.0 millimeters (mm) to about 9.0 millimeters (mm). In certain particular applications, the implanted height H of the intervertebral implant 10 may be no greater than 8.5 millimeters (mm). It will be understood that the implanted height H of the intervertebral implant 10 may be different than a height H_{F} associated with an assembled intervertebral implant 10, as the teeth 29 of the first bone engagement surface 28 and the second bone engagement surface 32 may bite into and be substantially received into the respective vertebral body 12. Further, the implant height H of the intervertebral implant 10 can be adjusted for optimal fit between the adjacent vertebral bodies 12, and the implant height H can depend upon the particular anatomical conditions of the patient. Thus, in certain instances, it may be desirable to provide a kit of various intervertebral implants 10, each having a distinct implant height H.

Turning to FIG. 6A, a cross-sectional view of an alternative example of an intervertebral implant 100 in accordance with the present teachings is illustrated. Given the similarities between the intervertebral implant 10 and the intervertebral implant 100, like reference numbers will be used to identify similar components and features. The intervertebral implant 100 differs from the intervertebral implant 10 in that a stop element 102 can extend from the first articulating surface 26 of the inferior component 18.

The stop element 102 can extend outwardly or downwardly from the first articulating surface 26, and can be received within the aperture 24 of the superior component 20. The stop element 102 can cooperate with a sidewall 24a of the aperture 24 to provide a stop that limits the range of motion or articulation between the inferior component 18 and the superior component 20. In certain instances, the stop element 102 can function as a camming element. In other words, when the inferior component 18 and the superior component 20 move or articulate through a selected range, the stop element 102 can engage the sidewall 24a of the aperture 24 to limit the further motion or articulation of the inferior component 18 and the superior component 20.

Generally, the stop element 102 can comprise a soft stop that increases resistance to continued articulation, instead of a hard stop (i.e., a stop that completely prevents further articulation). The soft stop provided by the stop element 102 can function to control the range of motion or articulation of the inferior component 18 and the superior component 20 at the extremes of full flexion/extension of the spine, full lateral bending of the spine, and maximum anterior/posterior translation of the spine. In general, the stop element 102 and the aperture 24 can be cooperatively configured such that the intervertebral implant 10 mimics normal anatomical motion. In one example, the stop element 102 and the aperture 24 can be cooperatively configured to provide an unimpeded range of motion of about ±22-25 degrees for flexion/extension, about ±15 degrees for lateral bending, and about ±1-3 millimeters (mm) for anterior/posterior translation.

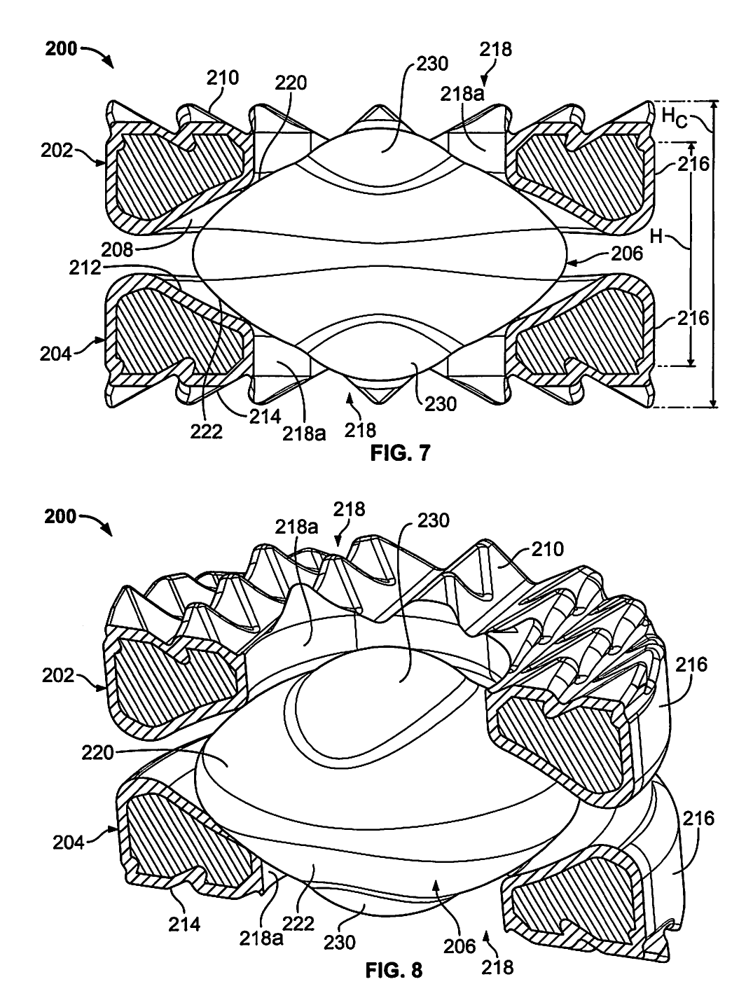

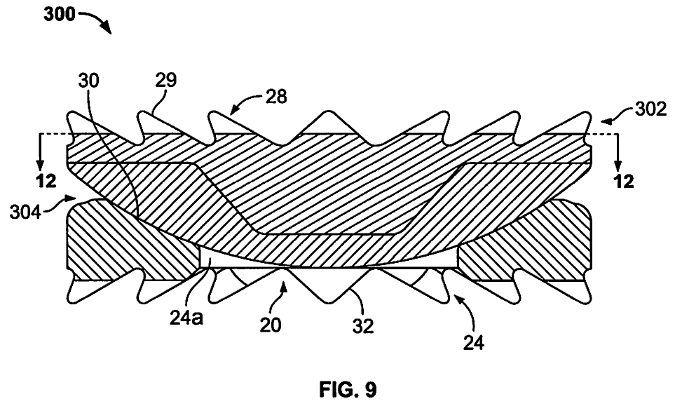

With reference to the cross-sectional views of FIGS. 7 and 8, another one of various intervertebral implants in accordance with the present teachings is illustrated and identified at reference character 200. The intervertebral implant 200 can generally include a first or inferior component 202 and a second or superior component 204. The intervertebral implant 200 can further include a third component or core 206 positioned between the inferior component 202 and the superior component 204. The intervertebral implant 200 will generally be understood to be a three-piece intervertebral implant, whereas the intervertebral implants 10, 100 will generally be understood to be two-piece intervertebral implants.

At least one of the inferior component 202 and the superior component 204 can have a generally toroidal shape. In the example illustrated, both the inferior component 202 and the superior component 204 can have a generally toroidal shape, as will be discussed further herein. The inferior component 202 and the superior component 204 can each be composed of any suitable biocompatible material, such as a biocompatible metal or polymer. For example, the inferior component 202 and the superior component 204 can be composed of titanium, cobalt chromium, stainless steel, pyrolytic carbon, etc. If desired, at least one of the inferior component 202 and the superior component 204 can be coated with a suitable biocompatible coating, such as an antibiotic, bone growth material, etc. It should be noted that although the inferior component 202 and the superior component 204 are described and illustrated herein as comprising a single integral component, the inferior component 202 and/or the superior component 204 could comprise multiple components coupled together, if desired. For example, the inferior component 202 and/or the superior component 204 could comprise a biocompatible polymer, such as polyethylene, coupled to a biocompatible metal, such as titanium, through a suitable technique. In addition, at least one of the inferior component 202 and the superior component 204 can constructed to include a shell and a core, such as the shell 34 and inner core 36 described with regard to FIGS. 5 and 6. The inferior component 202 and the superior component 204 can also be composed of substantially distinct materials, if desired.

In one example, the inferior component 202 and the superior component 204 can be substantially identical. As such, the remainder of this description will focus on the details of the inferior component 202, but a complete understanding of the superior component 204 will be readily apparent therefrom. It should be noted, however, that the inferior component 202 and the superior component 204 can be distinctly constructed.

The inferior component 202 can generally include a third articulating surface 208 and a first bone engagement surface 210. Thus, the superior component 204 can generally include a fourth articulating surface 212 and a second bone engagement surface 214. The first bone engagement surface 210 and the second bone engagement surface 214 can each engage a respective vertebra or one of the vertebral bodies 12. As the first bone engagement surface 210 and the second bone engagement surface 214 can be similar to the first bone engagement surface 28 described with reference to FIGS. 2-6, the first bone engagement surface 210 and the second bone engagement surface 214 will not be discussed in great detail herein. Briefly, however, the first bone engagement surface 210 and the second bone engagement surface 214 can each include the teeth 29, which can bite, alter or engage a respective one of the vertebral bodies 12.

With continued reference to FIGS. 7 and 8, the third articulating surface 208 and the fourth articulating surface 212 can be convex, concave or combinations thereof. In this example, the third articulating surface 208 and the fourth articulating surface 212 can be generally concave. In this regard, the third articulating surface 208 and the fourth articulating surface 212 can each be hemispherical and can each have a third radius of curvature. It should be noted, however, that the third articulating surface 208 and the fourth articulating surface 212 can have any shape that enables motion between the inferior component 202 and the superior component 204. For example, the third articulating surface 208 and the fourth articulating surface 212 can each include distinct radii of curvature that may or may not be concentric.

The inferior component 202 and superior component 204 can each be generally toroidal in shape. In this regard, each of the inferior component 202 and the superior component 204 can include a main body 216 that defines a substantially closed perimeter and an opening or aperture 218. The aperture 218 may be a central opening, as it is surrounded by the main body 216. It should be noted, however, that the term “central opening” does not narrowly mean that the aperture 218 must be centered relative to each of the inferior component 202 and the superior component 204. Rather, the aperture 218 can be offset from a central axis, if desired.

In one example, the aperture 218 can extend through each of the inferior component 202 and the superior component 204 from the respective articulating surfaces 208, 212 to the respective bone engagement surfaces 210, 214. Each aperture 218 can generally receive a portion of the core 206, which can reduce the implanted height H of the intervertebral implant 200. It will be understood, however, that each aperture 218 need not extend through the inferior component 202 and/or the superior component 204. In this regard, the aperture 218 can extend through only a portion of the inferior component 202 and/or the superior component 204. In the case of the aperture 218 extending only partially through the inferior component 202 and/or the superior component 204, the aperture 218 may intersect the second articulating surface 30, but may extend only substantially through the inferior component 202 and/or the superior component 204. In other words, the aperture 218 can be formed as a depression within the third articulating surface 208 and fourth articulating surface 212 so that the aperture 218 can receive a portion of the core 206 to reduce the implanted height H of the intervertebral implant 200, but the aperture 218 need not extend all the way through the inferior component 202 and/or the superior component 204.

The core 206 can articulate relative to at least one of the inferior component 202 and the superior component 204. In this example, the core 206 can articulate relative to both the inferior component 202 and the superior component 204. It should be understood that this is merely exemplary, as the core 206 can be fixed relative to one of the inferior component 202 and the superior component 204, if desired.

The core 206 can be composed of a suitable biocompatible material, such as a biocompatible metal or polymer. For example, the core 206 can be composed of titanium, cobalt chromium, stainless steel, pyrolytic carbon, graphite, polyethylene etc. If desired, the core 206 can be coated with a suitable biocompatible coating, such as an antibiotic, bone growth material, lubricant, etc. It should be noted that although the core 206 is described and illustrated herein as comprising a single integral component, the core 206 could comprise multiple components coupled together, if desired. For example, the core 206 could be composed of a shell and a core, similar to the shell 34 and inner core 36 discussed with regard to FIGS. 5 and 6. If the core 206 is composed of a shell and a core, in one example, the shell of the core 206 could comprise a biocompatible polymer, such as polyethylene, and the core could comprise a suitable biocompatible polymer or biocompatible metal having a reduced hardness.

In one example, the core 206 can be generally symmetrical about a horizontal mid-plane. The core 206 can include a fifth articulating surface 220 and a sixth articulating surface 222. The fifth articulating surface 220 can be generally opposite the sixth articulating surface 222. The fifth articulating surface 220 and the sixth articulating surface 222 can each cooperate with a respective one of the third articulating surface 208 and the fourth articulating surface 212 to enable relative motion between the inferior component 202, the superior component 204 and the core 206. The relative motion between the inferior component 202, the superior component 204 and the core 206 can enable relative motion between the adjacent vertebral bodies 12, thereby resulting in more natural motion of the spine of the patient. Thus, the fifth articulating surface 220 and the sixth articulating surface 222 can be of any shape to cooperate with the third articulating surface 208 and the fourth articulating surface 212 to permit relative motion between adjacent vertebral bodies 12.

In this example, as the third articulating surface 208 and the fourth articulating surface 212 can have a generally concave shape, the fifth articulating surface 220 and the sixth articulating surface 222 can have a generally convex shape. It should be noted, however, that the fifth articulating surface 220 and the sixth articulating surface 222 could comprise a generally concave shape, and the third articulating surface 208 and the fourth articulating surface 212 could comprise a generally convex shape, if desired. The use of cooperating or mating shapes between the third articulating surface 208 and the fourth articulating surface 212, and the fifth articulating surface 220 and the sixth articulating surface 222, can prevent expulsion of the core 206.

In this example, each of the fifth articulating surface 220 and the sixth articulating surface 222 can have a generally hemispherical surface, which can define a fourth radius of curvature. It should be noted, however, that the fifth articulating surface 220 and the sixth articulating surface 222 can have any shape that enables motion between the inferior component 202, the superior component 204 and the core 206. For example, the fifth articulating surface 220 and the sixth articulating surface 222 could each comprise distinct radii of curvature that may or may not be concentric.

In addition, as best shown in FIGS. 7 and 8, the fourth radius of curvature can be smaller than the third radius of curvature, which can establish line contact between the fifth articulating surface 220 and the sixth articulating surface 222 of the core 206 and the third articulating surface 208 and the fourth articulating surface 212 of the inferior component 202 and the superior component 204, respectively. The line contact may be generally arcuate due to the generally hemispherical surfaces of each of the inferior component 202, the superior component 204 and the core 206. The line contact between the inferior component 202, the superior component 204 and the core 206 can maintain stable articulation between the inferior component 202, the superior component 204 and the core 206.

Further, if the fourth radius of curvature associated with the core 206 is smaller than the third radius of curvature associated with the inferior component 202 and the superior component 204, a portion of the fifth articulating surface 220 and the sixth articulating surface 222 can extend into the aperture 218 defined in each of the inferior component 202 and the superior component 204. In this manner, the profile of the intervertebral implant 200 may be reduced without compromising the performance of the intervertebral implant 200.

With continued reference to FIGS. 7 and 8, the fifth articulating surface 220 and the sixth articulating surface 222 of the core 206 can include stop elements 230. As the stop elements 230 can be substantially similar to the stop element 102 described with regard to FIG. 6A, the stop elements 230 will not be described in great detail herein. Briefly, however, the stop elements 230 can extend at least partially into the respective apertures 218 of the inferior component 202 and the superior component 204. The stop elements 230 can cooperate with a sidewall 218a of each of the apertures 218 to provide a soft stop that limits the range of articulation between the inferior component 202 and the superior component 204. In this manner, the stop elements 230 function as camming elements.

In other words, when the inferior component 202 and the superior component 204 move or articulate through a predetermined range, the stop elements 230 can engage the sidewall 218a of a respective one of the apertures 218 to limit the further motion or articulation of the inferior component 202 and the superior component 204. Generally, the stop elements 230 can comprise a soft stop that increases resistance to continued articulation, instead of a hard stop (i.e., a stop that completely prevents further articulation). The soft stop provided by the stop elements 230 can function to control the range of motion or articulation of the inferior component 202 and the superior component 204 at the extremes of full flexion/extension of the spine, full lateral bending of the spine, and maximum anterior/posterior translation of the spine. In one example, the stop elements 230 and the apertures 218 can be cooperatively configured to provide an unimpeded range of motion of about ±22-25 degrees for flexion/extension, about ±15 degrees for lateral bending, and about ±1-3 millimeters (mm) for anterior/posterior translation. In addition, the stop elements 230 can act to prevent expulsion of the intervertebral implant 200.

The intervertebral implant 200 can be assembled by aligning the fifth articulating surface 220 and the sixth articulating surface 222 of the core 206 with the third articulating surface 208 and the fourth articulating surface 212 of the inferior component 202 and the superior component 204 such that the core 206 is at least partially received within the apertures 218 of the inferior component 202 and the superior component 204. As the intervertebral implant 200 can be inserted into the anatomy in the same manner described with regard to the intervertebral implant 10 of FIGS. 1-6, the insertion of the intervertebral implant 200 into the anatomy will not be discussed in great detail herein.

Once the intervertebral implant 200 is positioned between adjacent vertebral bodies 12, the implanted height H of the intervertebral implant 200 can be substantially similar to the height H_{D} of a healthy disc 16 so as to restore substantially normal function to the spine of the patient. In this example, the implanted height H of the assembled intervertebral implant 200 can be substantially similar to the implanted height H described with regard to the intervertebral implant 10, and thus, the specific implanted height H of the intervertebral implant 200 will not be discussed in great detail herein.

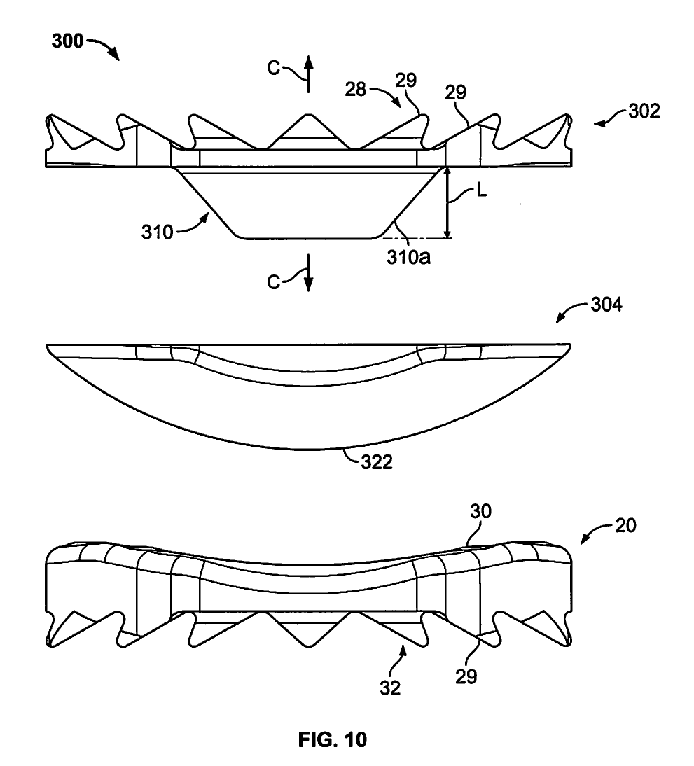

With reference now to FIGS. 9-12, in one example, an intervertebral implant 300 can be employed to repair a damaged portion of an anatomy. As the intervertebral implant 300 can be similar to the intervertebral implant 10 described with reference to FIGS. 1-6, only the differences between the intervertebral implant 10 and the intervertebral implant 300 will be discussed in great detail herein, and the same reference numerals will be used to denote the same or similar components.

With continued reference to FIGS. 9-12, the intervertebral implant 300 can include a first or inferior component 302, the second or superior component 20 and a third or articulation component 304. In this example, the intervertebral implant 300 can comprise a three-piece implant, with the articulation component 304 fixedly coupled to the inferior component 302, and positioned between the inferior component 302 and the superior component 20.

The inferior component 302 can be annular or generally ellipsoidal in shape, however, the inferior component 302 could be generally toroidal in shape, if desired. The inferior component 302 can comprise an integral component, which can be composed of a suitable biocompatible material, such as a biocompatible metal or polymer. For example, the inferior component 302 can be composed of titanium, cobalt chromium, stainless steel, pyrolytic carbon, etc. If desired, the inferior component 302 can be coated with a suitable biocompatible coating, such as an antibiotic, bone growth material, etc.

It should be noted that although the inferior component 302 is described and illustrated herein as comprising a single integral component, the inferior component 302 could comprise multiple components coupled together, if desired. For example, the inferior component 302 could comprise a biocompatible polymer, such as polyethylene, coupled to a biocompatible metal, such as titanium, through a suitable technique. As a further example, the inferior component 302 could be composed of a shell and a core, similar to the shell 34 and inner core 36 discussed with regard to FIGS. 5 and 6. If the inferior component 302 is composed of a shell and a core, in one example, the shell of the inferior component 302 could comprise pyrolytic carbon, and the core could comprise a suitable biocompatible polymer or biocompatible metal having a reduced hardness, such as graphite.

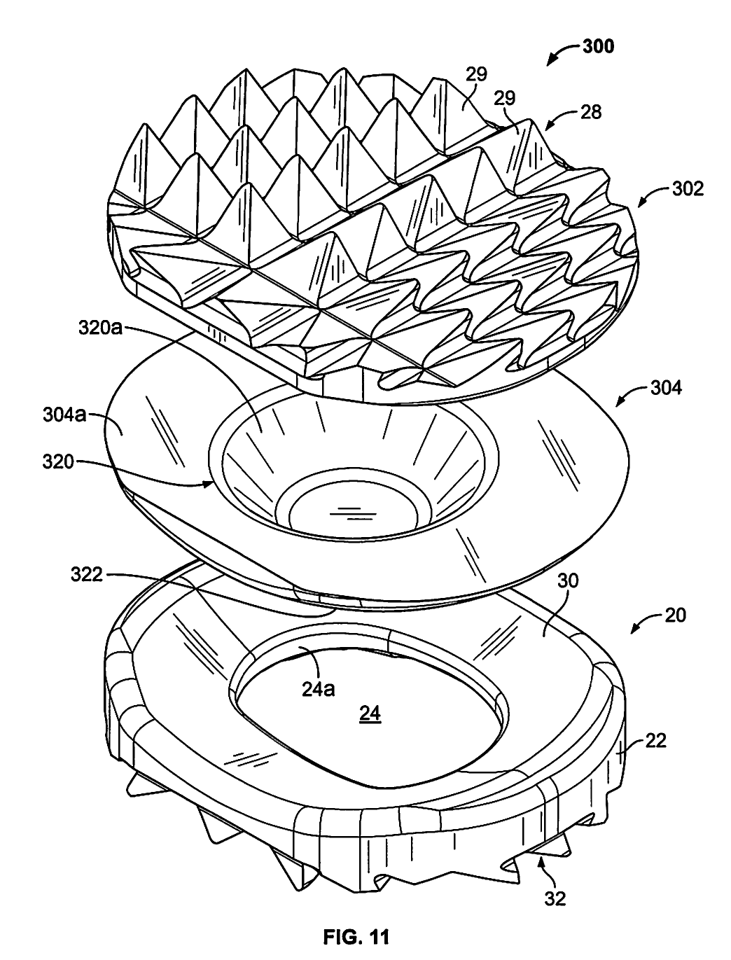

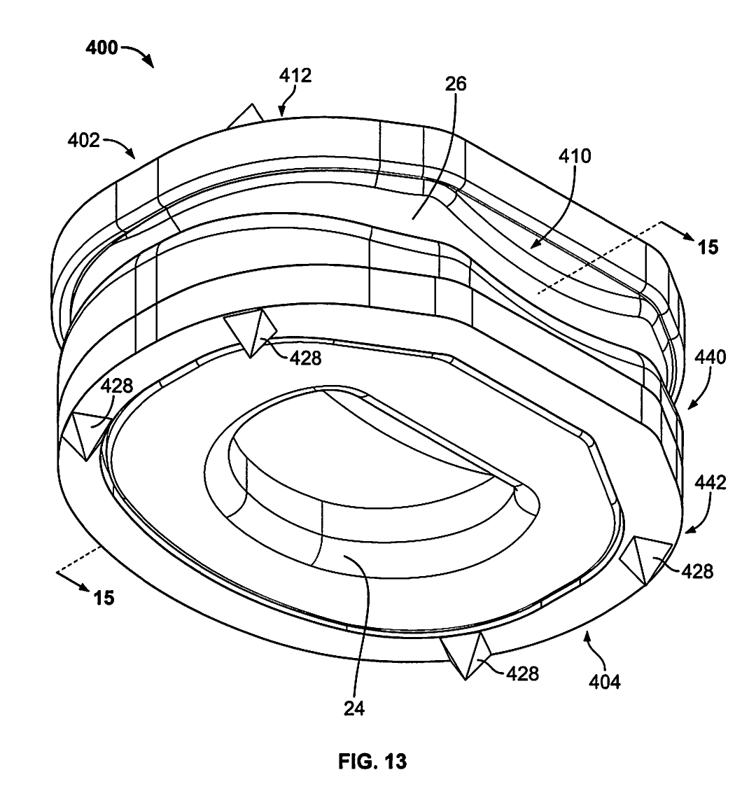

With reference to FIGS. 10-12, the inferior component 302 can include the first bone engagement surface 28 and a male connection or first mating portion 310. Generally, the first bone engagement surface 28 can be positioned opposite the first mating portion 310. In one example, the first mating portion 310 can comprise a projection formed about a central axis C of the intervertebral implant 300, however, the projection could be formed offset from the central axis C, if desired. As will be discussed in greater detail herein, the first mating portion 310 can cooperate with the articulation component 304 to enable relative motion between the inferior component 302 and the superior component 20 (FIG. 12). It should be noted that the use of a projection is merely exemplary, as any suitable technique could be used to couple the inferior component 302 to the articulation component 304, such as the use of adhesives, welding, snap-fit, etc.

With reference to FIG. 10, the first mating portion 310 can have a length L, which can extend an amount sufficient to couple the inferior component 302 to the articulation component 304. In addition, the first mating portion 310 can generally include a tapered surface 310a. As will be discussed, the tapered surface 310a of the projection of the first mating portion 310 can cooperate with a portion of the articulation component 304 to form an interference fit that can couple the inferior component 302 to the articulation component 304. It should be noted that the use of the tapered surface 310a is merely exemplary, as any suitable technique could be used to couple the inferior component 302 to the articulation component 304, such as the use of one or more circumferential sealing flanges, grooves, notches, keyed portions, mechanical fasteners, etc.

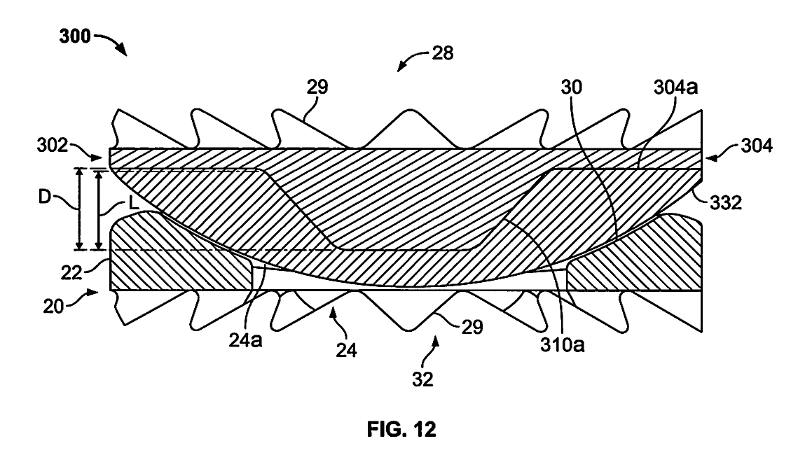

With reference to FIGS. 10-12, the articulation component 304 can include a female connection or second mating portion 320 (FIGS. 11 and 12) and a seventh articulating surface 322 (FIGS. 10 and 12). The articulation component 304 can be annular or generally ellipsoidal in shape, however, the articulation component 304 could be generally toroidal in shape, if desired. The articulation component 304 can comprise an integral component, which can be composed of a suitable biocompatible material, such as a biocompatible metal or polymer. For example, the articulation component 304 can be composed of titanium, cobalt chromium, stainless steel, pyrolytic carbon, polyethylene, etc. If desired, the articulation component 304 can be coated with a suitable biocompatible coating, such as an antibiotic, bone growth material, etc.

It should be noted that although the articulation component 304 is described and illustrated herein as comprising a single integral component, the articulation component 304 could comprise multiple components coupled together, if desired. For example, the articulation component 304 could comprise a biocompatible polymer, such as polyethylene, coupled to a biocompatible metal, such as titanium, through a suitable technique. As a further example, the articulation component 304 could be composed of a shell and a core, similar to the shell 34 and inner core 36 discussed with regard to FIGS. 5 and 6. If the articulation component 304 is composed of a shell and a core, in one example, the shell of the articulation component 304 could comprise pyrolytic carbon, and the core could comprise a suitable biocompatible polymer or biocompatible metal having a reduced hardness, such as graphite.

With reference to FIGS. 11 and 12, the second mating portion 320 can be opposite the seventh articulating surface 322, and can be configured to engage or mate with the mating portion 310 of the inferior component 302. Thus, in this example, the second mating portion 320 can comprise a bore, which can be formed about a central axis C of the intervertebral implant 300. It should be noted, however, that the bore could be formed offset from the central axis C, so long as the bore can cooperate with the first mating portion 310 to couple the articulation component 304 to the inferior component 302.

The second mating portion 320 can have a depth D, which can be at least as great as the length L of the first mating portion 310. As illustrated, the bore does not extend through the articulation component 304, however, it should be understood that the bore can extend through a proximal side 304a of the articulation component 304 to the seventh articulating surface 322, if desired. If the bore does extend through to the seventh articulating surface 322, the first mating portion 310 of the inferior component 302 can include a curvature so that the engagement of the inferior component 302 with the articulation component 304 does not substantially interrupt the seventh articulating surface 322. It should also be noted that the depth D of the second mating portion 320 and the length L of the first mating portion 310 can be varied to adjust of the implanted height H of the intervertebral implant 300.

In this example, with reference to FIGS. 11 and 12, the second mating portion 320 can include a tapered surface 320a, which can mate with the tapered surface 310a of the first mating portion 310 to couple the inferior component 302 to the articulation component 304. For example, the tapered surfaces 310a, 320a can define a morse taper, which can couple the inferior component 302 to the articulation component 304. It should be noted that the use of the tapered surface 320a is merely exemplary, as any suitable technique could be used to couple the first mating portion 310 to the second mating portion 320, such as the use of one or more circumferential sealing flanges, grooves, notches, keyed portions, mechanical fasteners, barbs, etc.

The seventh articulating surface 322 can be convex, concave or combinations thereof. In the example of FIGS. 9-12, the seventh articulating surface 322 can be generally convex. It should be noted, that although the seventh articulating surface 322 is illustrated and described herein as being generally convex, the seventh articulating surface 322 can have any shape that enables motion between the articulation component 304 and the superior component 20. Further, the seventh articulating surface 322 could comprise a generally concave shape, and the second articulating surface 30 could comprise a generally convex shape, if desired. Moreover, the seventh articulating surface 322 can include distinct radii of curvature that may or may not be concentric. Typically, the seventh articulating surface 322 can comprise any surface that can cooperate with the second articulating surface 30 to enable relative motion between the vertebral bodies 12. In this example, the seventh articulating surface 322 can be substantially hemispherical, and can include a fifth radius of curvature.

In one example, the second radius of curvature associated with the second articulating surface 30 of the superior component 20 can be greater than the fifth radius of curvature of the articulation component 304. The larger second radius of curvature of the superior component 20 can establish line contact between the seventh articulating surface 322 of the articulation component 304 and the second articulating surface 30 of the superior component 20. The line contact may be generally arcuate due to the generally hemispherical surfaces of each of the articulation component 304 and the superior component 20. The line contact between the articulation component 304 and the superior component 20 can maintain stable articulation between the articulation component 304 and the superior component 20.

Further, if the second radius of curvature associated with the superior component 20 is greater than the first radius of curvature associated with the articulation component 304, a portion of the seventh articulating surface 322 can extend into the aperture 24 of the superior component 20. In this manner, the profile of the intervertebral implant 300 may be reduced without compromising the performance of the intervertebral implant 300, as discussed with regard to the intervertebral implant 10.

The intervertebral implant 300 can be assembled by coupling the first mating portion 310 of the inferior component 302 to the second mating portion 320 of the articulation component 304. Then, the seventh articulating surface 322 can be aligned with the second articulating surface 30 of the superior component 20, such that the articulation component 304 is at least partially received within the aperture 24 of the superior component 20. As the intervertebral implant 300 can be inserted into the anatomy in the same manner described with regard to the intervertebral implant 10 of FIGS. 1-6, the insertion of the intervertebral implant 300 into the anatomy will not be discussed in great detail herein.

Once the intervertebral implant 300 is positioned between adjacent vertebral bodies 12, the implanted height H of the intervertebral implant 300 can be substantially similar to the height H_{D} of a healthy disc 16 so as to restore substantially normal function to the spine of the patient. In this example, the implanted height H of the assembled intervertebral implant 300 can be substantially similar to the implanted height H described with regard to the intervertebral implant 10, and thus, the specific implanted height H of the intervertebral implant 300 will not be discussed in great detail herein.

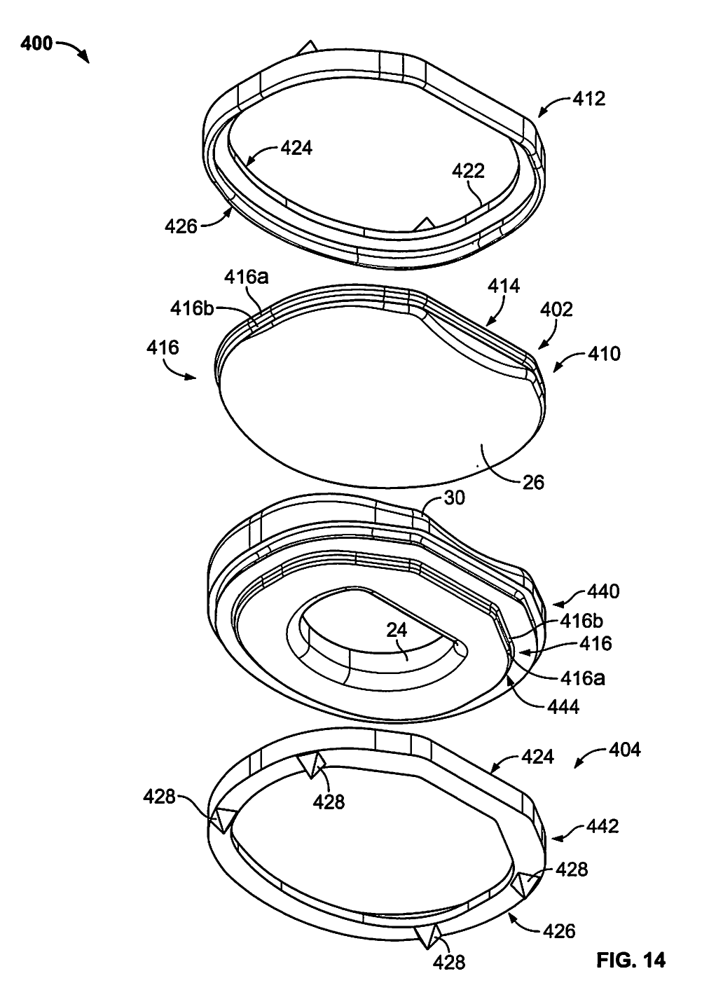

With reference now to FIGS. 13-15, in one example, an intervertebral implant 400 can be employed to repair a damaged portion of an anatomy. As the intervertebral implant 400 can be similar to the intervertebral implant 10 described with reference to FIGS. 1-6, only the differences between the intervertebral implant 10 and the intervertebral implant 400 will be discussed in great detail herein, and the same reference numerals will be used to denote the same or similar components. With continued reference to FIGS. 13-15, the intervertebral implant 400 can include a first or inferior component 402 and a second or superior component 404. As will be discussed, in this example, the intervertebral implant 400 can comprise a four-piece implant, with the inferior component 402 directly articulating with the superior component 404.

The inferior component 402 can comprise a two-piece component, and in this example, the inferior component 402 can include a body 410 and a bone engaging portion or inferior crown 412. It should be noted that although the inferior component 402 is described and illustrated herein as comprising a two-piece component, the inferior component 402 could comprise an integral component formed through a suitable processing technique, if desired.

The body 410 can be composed of a suitable biocompatible metal or polymer. For example, the body 410 can be composed of titanium, cobalt chromium, stainless steel, pyrolytic carbon, etc. In one example, the body 410 can be composed of the shell 34 and the core 36 discussed with regard to FIGS. 5 and 6. It should be noted, however, that the body 410 can be composed of any desired material, and could be composed of a single solid material, if desired. In addition, the body 410 can be coated with a suitable biocompatible coating, such as an antibiotic, bone growth material, etc.

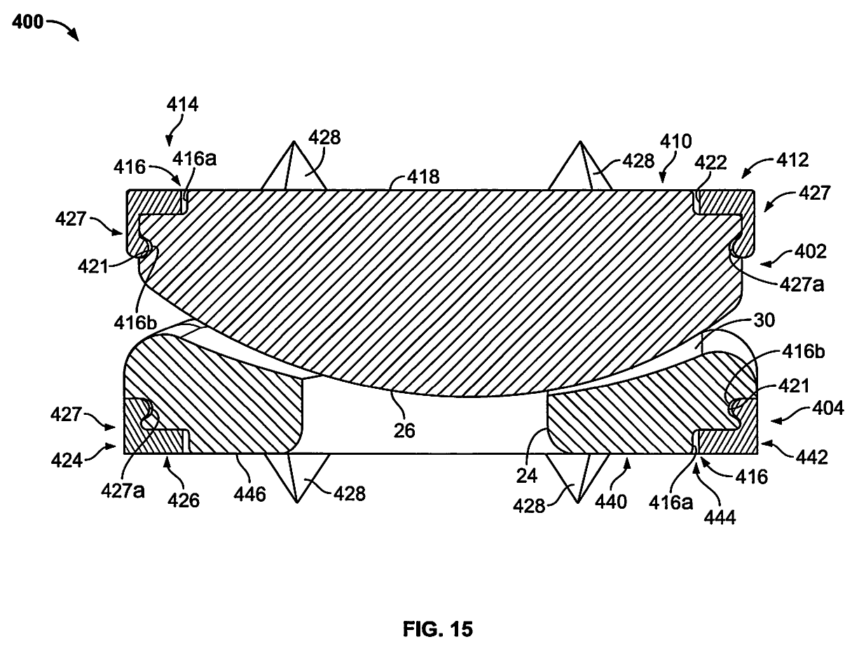

The body 410 can be annular or generally ellipsoidal in shape, however, the body 410 could be generally toroidal in shape, if desired. The body 410 can include the first articulating surface 26 and a first receiver portion or surface 414 (FIG. 15). As best shown in FIG. 15, the first receiver surface 414 can include at least one or a plurality of grooves 416, which can couple the inferior crown 412 to the body 410. The first receiver surface 414 can also define a bone contact surface 418, which can be disposed adjacent to or in contact with the spinous processes when the intervertebral implant 400 is coupled to the anatomy. The grooves 416 can generally be defined between the first articulating surface 26 and the bone contact surface 418.

In this example, the first receiver surface 414 can comprise two concentric grooves 416a, 416b. Each of the grooves 416 can have a thickness 420, which can each be different. In one example, the outermost groove 416a, which can be adjacent to the bone contact surface 418, can have a substantially planar cross-section, as illustrated in FIG. 15, while the groove 416b can have a substantially non-planar cross-section. In this regard, the groove 416b can define a recess 421, which can resist the separation of the inferior crown 412 from the body 410.

It should be understood, however, that the first receiver surface 414 can have any desired surface for mating with the inferior crown 412, such as notched, tapered, keyed, etc. Further, the first receiver surface 414 could be substantially planar, and the inferior crown 412 could be coupled to the first receiver surface 414 via a biocompatible adhesive. As another alternative, the first receiver surface 414 could include one or more apertures, which could receive mating projections on the inferior crown 412 or mechanical fasteners, to couple the inferior crown 412 to the body 410.

Thus, the inferior crown 412 can be coupled to the body 410 through any suitable technique, and optionally, could be integrally formed with the body 410. The inferior crown 412 can be composed of a suitable biocompatible metal or polymer. For example, the inferior crown 412 can be composed of titanium, cobalt chromium, stainless steel, pyrolytic carbon, etc. It should be noted, however, that the inferior crown 412 can be composed of any desired material, and could be composed of a two different materials as discussed with regard to the shell 34 and the core 36 of FIGS. 5 and 6. In addition, the inferior crown 412 can be coated with a suitable biocompatible coating, such as an antibiotic, bone growth material, etc. It should be noted, however, that in the example of the body 410 being formed of a shell 34 and core 36, the inferior crown 412 can generally be formed of a single solid material, such as titanium.

The inferior crown 412 can be configured to engage the first vertebra or vertebral body 12a to couple the inferior component 402 to the anatomy. In this regard, with reference to FIGS. 13-15, the inferior crown 412 can be generally annular, and in this example, can be ring-like and can define an aperture 422. The inferior crown 412 can include a first mating portion or surface 424 and a bone engagement portion or surface 426 disposed about the periphery of the aperture 422.

As best shown in FIG. 15, the first mating surface 424 can be generally opposite the bone engagement surface 426, and can include a projection 427. The first mating surface 424 can be cooperate with the first receiver surface 414 to couple the inferior crown 412 to the body 410. In one example, the projection 427 can include a lip 427a, which can mate with the recess 421 of the groove 416b to couple the inferior crown 412 to the body 410 through a snap-fit engagement. Generally, the inferior crown 412 can be coupled to the body 410 such that the bone contact surface 418 of the body 410 is planar with at least a portion of the first bone engagement surface 426 of the inferior crown 412, and the lip 427a of the first mating surface 424 is fully seated within or received within the recess 421 of the groove 416b (FIG. 15).

The first bone engagement surface 426 can engage a first vertebra or vertebral body 12a, similar to the first bone engagement surface 28 discussed with regard to FIG. 1. The first bone engagement surface 426 can be configured in any manner well known in the art to resist expulsion of the intervertebral implant 400 from between the adjacent vertebral bodies 12, and to enable the inferior component 402 to self-center or self-align relative to the vertebral body 12a.

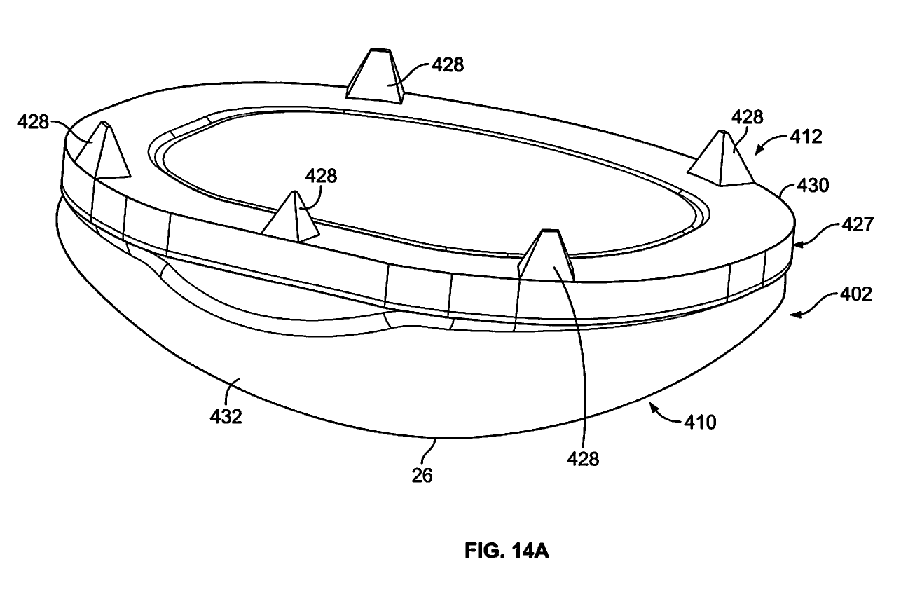

In one example, with reference to FIG. 14A, the first bone engagement surface 426 can include one or more aggressive multi-angled and self-centering teeth 428 for fixation. The teeth 428 can be extend outwardly from the inferior crown 412, and can be arranged in any desired configuration. As illustrated, in this example, the first bone engagement surface 426 can include five teeth 428. Two of the teeth 428 can be positioned near a first end 430 of the inferior crown 412 and three of the teeth 428 can be positioned near a second end 432 of the inferior crown 412, generally or substantially opposite the teeth 428 near the first end 430. Each of the teeth 428 of the first bone engagement surface 28 can each include an elongate angled surface, which can terminate at a distal point. The distal point can bite into or alter the surface of the vertebral body 12a to couple or fix the inferior crown 412 to the vertebral body 12a.

In one example, the various elongate angled surfaces of the teeth 428 can be arranged so as to enable the inferior crown 412 to self-center under loads from the adjacent vertebral bodies 12, as discussed with regard to the teeth 29. In addition, the inferior crown 412 can include a sub-plurality of teeth 428, which can prevent the expulsion of the inferior component 402. It should be noted, however, that the first bone engagement surface 426 can include any suitable bone engagement surface known in the art, such as spikes, barbs, etc.

With reference to FIGS. 13-15, the superior component 404 can comprise a two-piece component, and in this example, the superior component 404 can include a body 440 and a bone engaging portion or superior crown 442. It should be noted that although the superior component 404 is described and illustrated herein as comprising a two-piece component, the superior component 404 could comprise an integral component formed through a suitable processing technique, if desired.

The body 440 can be composed of a suitable biocompatible metal or polymer. For example, the body 440 can be composed of titanium, cobalt chromium, stainless steel, pyrolytic carbon, etc. In one example, the body 440 can be composed of the shell 34 and the core 36 discussed with regard to FIGS. 5 and 6. It should be noted, however, that the body 440 can be composed of any desired material, and could be composed of a single solid material, if desired. In addition, the body 440 can be coated with a suitable biocompatible coating, such as an antibiotic, bone growth material, etc.

It should be noted that the superior component 404 can be composed of the same material as the inferior component 402, or can be composed of a different material than the inferior component 402, depending upon desired strength properties, wear properties, etc. The body 440 can be generally toroidal in shape, and can include the second articulating surface 30, the aperture 24 and a second receiver surface 444.

As discussed, the second articulating surface 30 can have a generally hemispherical surface, which can define a second radius of curvature. It should be noted, however, that the second articulating surface 30 can have any shape that enables motion between the inferior component 402 and the superior component 404. For example, the second articulating surface 30 could comprise distinct radii of curvature that may or may not be concentric. In addition, as discussed with regard to FIGS. 5 and 6, the second radius of curvature of the superior component 404 can be greater than the first radius of curvature of the inferior component 402, which can establish line contact between the first articulating surface 26 and the second articulating surface 30. The line contact may be generally arcuate due to the generally hemispherical surfaces of each of the inferior component 402 and the superior component 404. The line contact between the inferior component 402 and the superior component 404 can maintain stable articulation between the inferior component 402 and the superior component 404.

Further, as discussed, if the second radius of curvature associated with the superior component 404 is greater than the first radius of curvature associated with the inferior component 402, a portion of the first articulating surface 26 can extend into the aperture 24 of the superior component 404. In this manner, the profile of the intervertebral implant 400 may be reduced without compromising the performance of the intervertebral implant 400.

With reference to FIG. 15, the second receiver surface 444 can be similar to the first receiver surface 414, and can include the at least one or plurality of grooves 416. The at least one or plurality of grooves 416 can couple the superior crown 442 to the body 440. The second receiver surface 444 can also define a bone contact surface 446, which can be disposed adjacent to or in contact with the spinous processes when the intervertebral implant 400 is coupled to the anatomy.

The grooves 416 can generally be defined between the second articulating surface 30 and the bone contact surface 446. It should be understood, however, that the second receiver surface 444 can have any desired surface for mating with the superior crown 442, such as notched, tapered, keyed, etc. Further, the second receiver surface 444 could be substantially planar, and the superior crown 442 could be coupled to the second receiver surface 444 via a biocompatible adhesive. As another alternative, the second receiver surface 444 could include one or more apertures, which could receive mating projections on the superior crown 442 or mechanical fasteners, to couple the superior crown 442 to the body 440.

Thus, the superior crown 442 can be coupled to the body 440 through any suitable technique, and optionally, could be integrally formed with the body 440. The superior crown 442 can be composed of a suitable biocompatible metal or polymer. For example, the superior crown 442 can be composed of titanium, cobalt chromium, stainless steel, pyrolytic carbon, etc. It should be noted, however, that the superior crown 442 can be composed of any desired material, and could be composed of a two different materials as discussed with regard to the shell 34 and the core 36 of FIGS. 5 and 6. In addition, the superior crown 442 can be coated with a suitable biocompatible coating, such as an antibiotic, bone growth material, etc. It should be noted, however, that in the example of the body 440 being formed of a shell 34 and core 36, the superior crown 442 can generally be formed of a single solid material, such as titanium.

The superior crown 442 can be configured to engage the second vertebra or vertebral body 12b to couple the superior component 404 to the anatomy. In one example, the superior crown 442 can be substantially similar to the inferior crown 412, and can include the first mating surface 424 and the bone engagement surface 426 disposed about the periphery of the aperture 422. The bone engagement surface 426 of the superior crown 442 can engage the second vertebra or vertebral body 12b, similar to the second bone engagement surface 32 discussed with regard to FIG. 1. It should be noted, however, that the superior crown 442 could be different from the inferior crown 412, if desired, and could include more or less teeth 428, for example, as illustrated.

Generally, the superior crown 442 can be shaped to correspond to the shape of the body 440 of the superior component 404, and the superior crown 442 can be coupled to the body 440 such that the bone contact surface 446 of the body 440 is planar with at least a portion of the bone engagement surface 426 of the superior crown 442, and the lip 427a of the first mating surface 424 is fully seated within or received within the recess 421 of the groove 416b.

The intervertebral implant 400 can be assembled by coupling or snapping the inferior crown 412 onto the inferior component 402, and coupling or snapping the superior crown 442 onto the superior component 404. Then, the first articulating surface 26 can be aligned with the second articulating surface 30 of the superior component 20, such that first articulating surface 26 is at least partially received within the aperture 24 of the superior component 404. As the intervertebral implant 400 can be inserted into the anatomy in the same manner described with regard to the intervertebral implant 10 of FIGS. 1-6, the insertion of the intervertebral implant 400 into the anatomy will not be discussed in great detail herein.

Once the intervertebral implant 400 is positioned between adjacent vertebral bodies 12, the implanted height H of the intervertebral implant 400 can be substantially similar to the height H_{D} of a healthy disc 16 so as to restore substantially normal function to the spine of the patient. In this example, the implanted height H of the assembled intervertebral implant 400 can be substantially similar to the implanted height H described with regard to the intervertebral implant 10, and thus, the specific implanted height H of the intervertebral implant 400 will not be discussed in great detail herein.

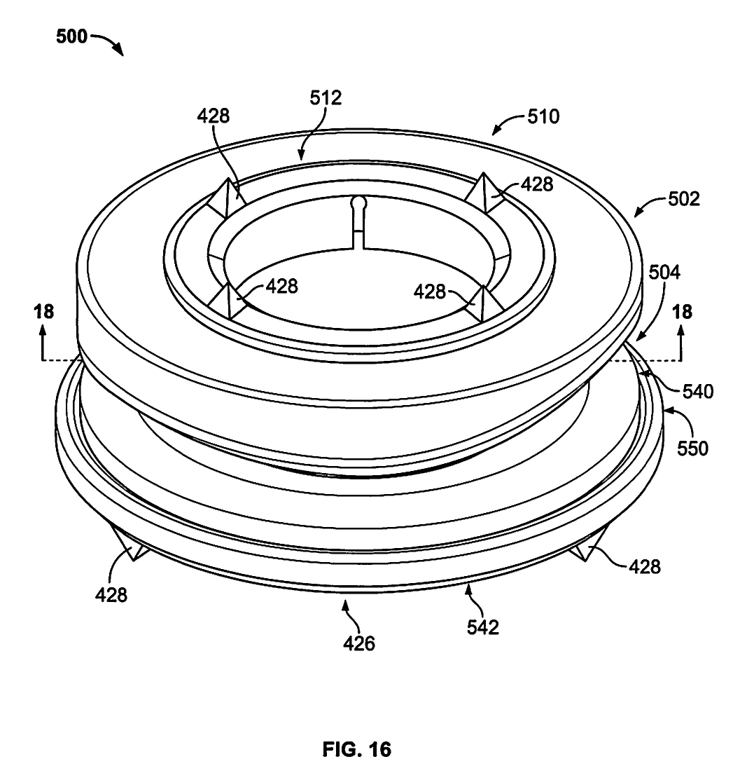

With reference now to FIGS. 16-18, in one example, an intervertebral implant 500 can be employed to repair a damaged portion of an anatomy. As the intervertebral implant 500 can be similar to the intervertebral implant 400 described with reference to FIGS. 13-15, only the differences between the intervertebral implant 400 and the intervertebral implant 500 will be discussed in great detail herein, and the same reference numerals will be used to denote the same or similar components. With continued reference to FIGS. 16-18, the intervertebral implant 500 can include a first or inferior component 502 and a second or superior component 504. As will be discussed, in this example, the intervertebral implant 500 can comprise a four-piece implant, with the inferior component 502 directly articulating with the superior component 504.

The inferior component 502 can comprise a two-piece component, and in this example, the inferior component 502 can include a body 510 and a bone engaging portion or inferior crown 512. It should be noted that although the inferior component 502 is described and illustrated herein as comprising a two-piece component, the inferior component 502 could comprise an integral component formed through a suitable processing technique, if desired.

The body 510 can be composed of a suitable biocompatible metal or polymer. For example, the body 510 can be composed of titanium, cobalt chromium, stainless steel, pyrolytic carbon, etc. In one example, the body 510 can be composed of the shell 34 and the core 36 discussed with regard to FIGS. 5 and 6. It should be noted, however, that the body 510 can be composed of any desired material, and could be composed of a single solid material, if desired. In addition, the body 510 can be coated with a suitable biocompatible coating, such as an antibiotic, bone growth material, etc.

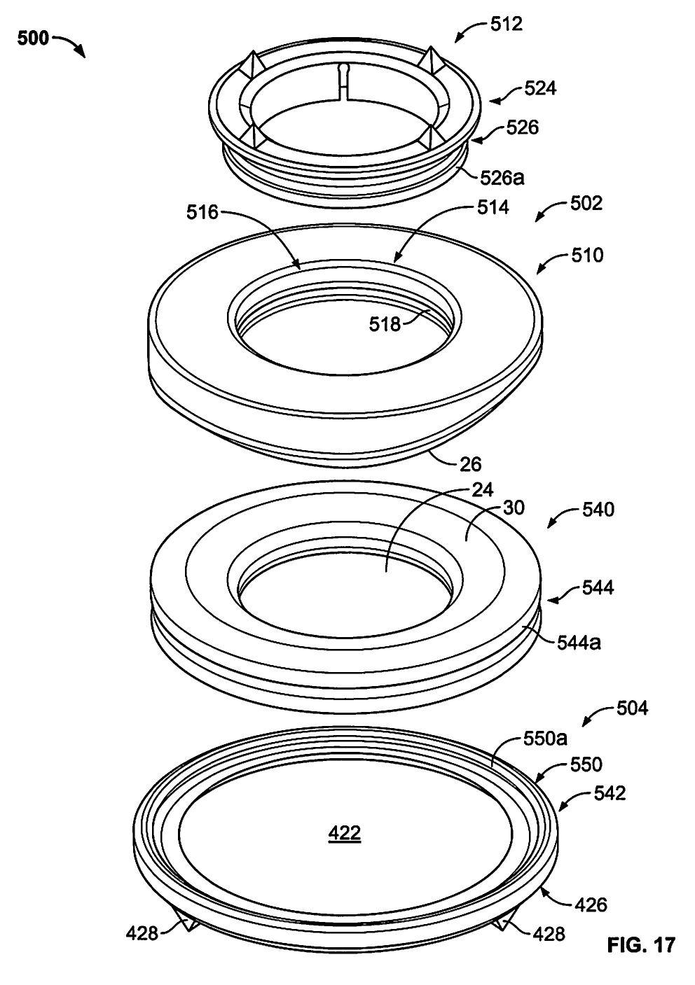

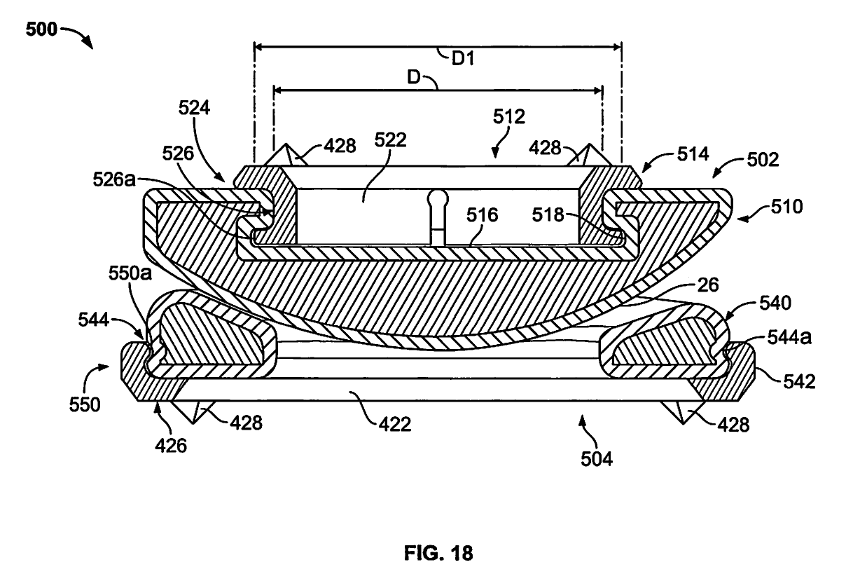

The body 510 can be annular or generally ellipsoidal in shape, however, the body 510 could be generally toroidal in shape, if desired. With reference to FIGS. 17 and 18, the body 510 can include the first articulating surface 26 and a first receiver surface 514. The first receiver portion or surface 514 can define a bore 516, which can receive a portion of the inferior crown 512 to couple the inferior crown 512 to the body 510.

In this regard, in one example, the bore 516 can be generally annular or cylindrical, and can generally be defined opposite the first articulating surface 26. The bore 516 can also define a groove 518. With reference to FIG. 18, the groove 518 can be formed substantially about a circumference of the bore 516, and can have a diameter D1, which can be greater than a diameter D of the bore 516. The diameter D1 of the groove 518 and the diameter D of the bore 516 can cooperate to enable the inferior crown 512 to be snap-fit into the body 510. It should be noted, however, that any suitable technique can be used to couple the inferior crown 512 to the body 510, such as mechanical fasteners, press-fit, welding, adhesives, riveting, etc. Optionally, the inferior crown 512 could be integrally formed with the body 510.

The inferior crown 512 can be composed of a suitable biocompatible metal or polymer. For example, the inferior crown 512 can be composed of titanium, cobalt chromium, stainless steel, pyrolytic carbon, etc. It should be noted, however, that the inferior crown 512 can be composed of any desired material, and could be composed of a two different materials as discussed with regard to the shell 34 and the core 36 of FIGS. 5 and 6. In addition, the inferior crown 512 can be coated with a suitable biocompatible coating, such as an antibiotic, bone growth material, etc. It should be noted, however, that in the example of the body 510 being formed of a shell 34 and core 36, the inferior crown 512 can generally be formed of a single solid material, such as titanium.

The inferior crown 512 can be configured to engage the first vertebra or vertebral body 12a to couple the inferior component 502 to the anatomy. In this regard, with reference to FIGS. 16-18, the inferior crown 512 can be generally annular, and in this example, can be ring-like and can define an aperture 522. It should be noted, however, that the aperture 522 can be optional, and the inferior crown 512 could be disc-like. The inferior crown 512 can include a first mating surface 524 and the bone engagement surface 426 disposed about the periphery of the aperture 522.

The first mating surface 524 can be generally opposite the bone engagement surface 426, and can include a flange 526. The first mating surface 524 can be cooperate with the first receiver surface 514 to couple the inferior crown 512 to the body 510. In one example, the flange 526 can include a lip 526a, which can mate with the groove 518 to couple the inferior crown 512 to the body 510 through a snap-fit engagement. Generally, the inferior crown 512 can be coupled to the body 510 such that the lip 526a is fully seated within or received within the groove 518.

The superior component 504 can comprise a two-piece component, and in this example, the superior component 504 can include a body 540 and a bone engaging portion or superior crown 542. It should be noted that although the superior component 504 is described and illustrated herein as comprising a two-piece component, the superior component 504 could comprise an integral component formed through a suitable processing technique, if desired.

The body 540 can be composed of a suitable biocompatible metal or polymer. For example, the body 540 can be composed of titanium, cobalt chromium, stainless steel, pyrolytic carbon, etc. In one example, the body 540 can be composed of the shell 34 and the core 36 discussed with regard to FIGS. 5 and 6. It should be noted, however, that the body 540 can be composed of any desired material, and could be composed of a single solid material, if desired. In addition, the body 540 can be coated with a suitable biocompatible coating, such as an antibiotic, bone growth material, etc.

It should be noted that the superior component 504 can be composed of the same material as the inferior component 502, or can be composed of a different material than the inferior component 502, depending upon desired strength properties, wear properties, etc. The body 540 can be generally toroidal in shape, and can include the second articulating surface 30, the aperture 24 and a second receiver surface 544.

The second receiver surface 544 can be defined about a portion of the body 540 generally opposite the second articulating surface 30. In one example, the second receiver surface 544 can be formed about a periphery or circumference of the body 540. In this example, the second receiver surface 544 can comprise a groove 544a, which can receive a portion of the superior crown 542 to couple the superior crown 542 to the body 540. It should be noted, however, that any suitable technique could be used to couple the superior crown 542 to the body 540, such as mechanical fasteners, press-fit, welding, adhesives, riveting, etc. Optionally, the superior crown 542 could be integrally formed with the body 540.

The superior crown 542 can be composed of a suitable biocompatible metal or polymer. For example, the superior crown 542 can be composed of titanium, cobalt chromium, stainless steel, pyrolytic carbon, etc. It should be noted, however, that the superior crown 542 can be composed of any desired material, and could be composed of a two different materials as discussed with regard to the shell 34 and the core 36 of FIGS. 5 and 6. In addition, the superior crown 542 can be coated with a suitable biocompatible coating, such as an antibiotic, bone growth material, etc. It should be noted, however, that in the example of the body 540 being formed of a shell 34 and core 36, the superior crown 542 can generally be formed of a single solid material, such as titanium.

The superior crown 542 can be configured to engage the second vertebra or vertebral body 12b to couple the superior component 504 to the anatomy. In one example, the superior crown 542 can include the aperture 422, a second mating surface 550 and the bone engagement surface 426. The bone engagement surface 426 can each be disposed about the aperture 422, while the second mating surface 550 can be defined about the periphery of the superior crown 542.

In this regard, the second mating surface 550 can define a lip 550a, which can extend outwardly from the superior crown 542 in a direction generally opposite the teeth 428 of the bone engagement surface 426. The lip 550a can be sized to engage the groove 544a of the second receiver surface 544 to couple the superior crown 542 to the body 540 (FIG. 18). Thus, when coupled to the body 540, the superior crown 542 can substantially surround an end of the body 540 and the superior crown 542 can substantially define the bone contact surface.

The intervertebral implant 500 can be assembled by coupling or snapping the inferior crown 512 onto the inferior component 502, and coupling or snapping the superior crown 542 onto the superior component 504. In this regard, the lip 526a of the inferior crown 512 can be coupled to or snapped into engagement with the groove 518 of the body 510 and the lip 550a of the superior crown 542 can be coupled or snapped into engagement with the groove 544a of the body 540. Then, the first articulating surface 26 can be aligned with the second articulating surface 30 of the superior component 20, such that first articulating surface 26 is at least partially received within the aperture 24 of the superior component 504. As the intervertebral implant 500 can be inserted into the anatomy in the same manner described with regard to the intervertebral implant 10 of FIGS. 1-6, the insertion of the intervertebral implant 500 into the anatomy will not be discussed in great detail herein.

Once the intervertebral implant 500 is positioned between adjacent vertebral bodies 12, the implanted height H of the intervertebral implant 500 can be substantially similar to the height H_{D} of a healthy disc 16 so as to restore substantially normal function to the spine of the patient. In this example, the implanted height H of the assembled intervertebral implant 500 can be substantially similar to the implanted height H described with regard to the intervertebral implant 10, and thus, the specific implanted height H of the intervertebral implant 500 will not be discussed in great detail herein.

Accordingly, the intervertebral implant 10, 100, 200, 300, 400, 500 can be used to repair damaged tissue in the anatomy, such as in the case of degenerative disc disease, via the insertion of the intervertebral implant 10, 100, 200, 300, 400, 500 between adjacent vertebral bodies 12. The generally toroidal shape of the superior component 20, 204, 404, 504 can enable the intervertebral implant 10, 100, 200, 300, 400, 500 to have the implanted height H, which can be substantially similar to the height H_{D} of a healthy disc 16 so as to restore substantially normal function to the spine of the patient. Further, the first bone engagement surface 28, 210, second bone engagement surface 32, 214 and bone engagement surface 426 can allow the intervertebral implant 10, 100, 200, 300, 400, 500 to automatically center under loads or forces applied by the vertebral bodies 12.

While specific examples have been described in the specification and illustrated in the drawings, it will be understood by those of ordinary skill in the art that various changes can be made and equivalents can be substituted for elements thereof without departing from the scope of the present teachings. Furthermore, the mixing and matching of features, elements and/or functions between various examples is expressly contemplated herein so that one of ordinary skill in the art would appreciate from the present teachings that features, elements and/or functions of one example can be incorporated into another example as appropriate, unless described otherwise, above. Moreover, many modifications can be made to adapt a particular situation or material to the present teachings without departing from the essential scope thereof. Therefore, it is intended that the present teachings not be limited to the particular examples illustrated by the drawings and described in the specification, but that the scope of the present teachings will include any embodiments falling within the foregoing description.

For example, while the intervertebral implant 300 has been described herein as having a substantially smooth seventh articulating surface 322, those of skill in the art will appreciate that the present disclosure, in its broadest aspects, may be constructed somewhat differently. In this regard, the intervertebral implant 300 can include the stop element 102, which can extend from the seventh articulating surface 322 of the articulation component 304. The stop element 102 can extend downwardly from the seventh articulating surface 322, and can be received within the aperture 24 of the superior component 20. The stop element 102 can cooperate with the sidewall 24a of the aperture 24 to limit the range of motion or articulation between the inferior component 302 and the superior component 20, as discussed with regard to FIG. 6A.

What is claimed is:

1. An intervertebral implant comprising:
a first component including a first articulating surface, the first articulating surface being generally convex with a first radius of curvature and defining a stop element; and
a second component defining an aperture and including a second articulating surface, the second articulating surface being generally concave having a second radius of curvature, the second articulating surface being articulable with the first articulating surface for retaining motion between the first and second vertebra,
wherein a portion of the first articulating surface including the stop element extends into the aperture and contacts a sidewall of the aperture to limit the range of articulation of the first component and the second component.

2. The intervertebral implant of claim 1, wherein the first articulating surface and the second articulating surface directly articulate relative to one another.

3. The intervertebral implant of claim 2, wherein the first component and the second component define a first vertebral engaging portion and a second vertebral engaging portion, respectively.

4. The intervertebral implant of claim 1, further comprising a third component, the first component defining a core disposed between the second component and the third component.

5. The intervertebral implant of claim 4, wherein the core further comprises a third articulating surface positioned opposite the first articulating surface and the third articulating surface is generally convex.

6. The intervertebral implant of claim 5, wherein the third component is generally in the shape of a toroid and further comprises a fourth articulating surface and a first bone engaging portion for engaging the first vertebra, the fourth articulating surface being generally concave and articulable with the third articulating surface for retaining motion between the first vertebra and the second vertebra.

7. The intervertebral implant of claim 5, wherein the third articulating surface further comprises a stop element to limit the range of motion between the first vertebra and the second vertebra.

8. The intervertebral implant of claim 5, wherein the fourth articulating surface has a larger radius of curvature than the third articulating surface such that a portion of the third articulating surface extends into an opening defined by the generally toroid shape of the third component.

9. An intervertebral implant comprising:
a first component having a first articulating surface, the first articulating surface being generally convex;
a second component defining an aperture and having a second articulating surface and a first bone engaging portion for engaging a first vertebra, the second articulating surface being generally concave and articulable with the first articulating surface for retaining motion between the first vertebra and a second vertebra; and
a third component coupled to the first component opposite the first articulating surface, the third component defining a second bone engaging portion for engaging the second vertebra,
wherein the second articulating surface has a larger radius of curvature than the first articulating surface such that a portion of the first articulating surface extends into the aperture of the second component.

10. The intervertebral implant of claim 9, wherein the third component further comprises a first mating portion that mates with a second mating portion of the first component to couple the third component to the first component to retain motion between the first vertebra and the second vertebra.

11. The intervertebral implant of claim 10, wherein the first mating portion and the second mating portion each include cooperating tapered surfaces.

12. The intervertebral implant of claim 9, wherein the third component further comprises a ring having a first mating portion opposite the second bone engaging portion that engages a first receiving portion defined on the first component to couple the third component to the first component.

13. The intervertebral implant of claim 12, further comprising a fourth component coupled to the second component opposite the second articulating surface that defines the first bone engaging portion.

14. The intervertebral implant of claim 13, wherein the fourth component further comprises a ring having a first mating portion opposite the first bone engaging portion that engages a second receiving portion defined on the second component to couple the fourth component to the first component.

15. The intervertebral implant of claim 14, wherein the third component and the fourth component are composed of a biocompatible metal and the first component and the second component are composed of a biocompatible polymer.

16. The intervertebral implant of claim 15, wherein the third component and the fourth component are composed of titanium.

17. The intervertebral implant of claim 15, wherein the first component and the second component are each composed of an outer shell formed of a first material having a first hardness and an inner core formed of a second material having a second hardness, the second hardness being different than the first hardness.

18. The intervertebral implant of claim 17, wherein the outer shell is formed from a pyrolytic carbon and the inner core is formed from graphite.

19. An intervertebral implant comprising:
a first component having a first articulating surface, the first articulating surface being generally convex;
a second component in the shape of a toroid defining an aperture and having a second articulating surface, the second articulating surface being generally concave and articulable with the first articulating surface for retaining motion between a first vertebra and a second vertebra;
a third component coupled to the first component opposite the first articulating surface, the third component defining a first bone engaging portion for engaging a first vertebra; and
a fourth component coupled to the second component opposite the second articulating surface so as to be disposed about a periphery of the aperture, the fourth component defining a second bone engaging portion,
wherein the second articulating surface has a larger radius of curvature than the first articulating surface such that a portion of the first articulating surface extends into the aperture of the second component.

20. The intervertebral implant of claim 19, wherein the first component defines a bore opposite the first articulating surface, and the third component is coupled to the first component via the bore.

21. The intervertebral implant of claim 19, wherein the third component and the fourth component are ring-shaped.
